(19) United States
(12) Patent Application Publication
(10) Pub. No.: US 2018/0360328 A1
(43) Pub. Date: Dec. 20, 2018

(54) BLOOD PRESSURE MEASUREMENT UTILIZING A FINGER CUFF IN CONJUNCTION WITH HEAT

(71) Applicant: Edwards Lifesciences Corporation, Irvine, CA (US)

(72) Inventors: Peiyuan Li, Amsterdam (NL); Hendrik Petrus Van Der Weij, Helmund (NL); Jacobus Jozef Gerardus Maria Settels, De Hoef (NL); Max Desiré Leonard Stotijn, Hoogeveen (NL)

(21) Appl. No.: 15/997,384

(22) Filed: Jun. 4, 2018

Related U.S. Application Data
(60) Provisional application No. 62/520,164, filed on Jun. 15, 2017.

(51) Int. Cl. A61B 5/022; A61B 5/00; A61B 5/1491

(52) U.S. Cl. CPC A61B 5/02241; A61B 5/0053; A61B 5/02141; A61B 5/1491; A61B 5/0059; A61B 5/6826

(57) ABSTRACT

Disclosed is a method to measure a patient's blood pressure by a blood pressure measurement system utilizing a finger cuff, in which the finger cuff includes a light emitting diode (LED)—photodiode (PD) pair and a bladder. The method comprises: placing the finger cuff around the patient's finger such that the bladder and the LED-PD pair aid in measuring the patient's blood pressure by the blood pressure measurement system; and applying heat generated by a heating element to the finger of the patient to increase the temperature of the finger, wherein the heating element generates heat at a predetermined temperature. Also, in some embodiments, the heating element also includes warming the hand and the wrist.

### CROSS-REFERENCE TO RELATED APPLICATIONS

This applications claims priority to U.S. Provisional Patent Application No. 62/520,164, filed Jun. 15, 2017, the contents of which is incorporated herein in its entirety.

### BACKGROUND

### Field

Embodiments of the invention relate generally to non-invasive blood pressure measurement. More particularly, embodiments of the invention relate to non-invasive blood pressure measurement using a finger cuff in conjunction with heat.

### Relevant Background

Volume clamping is a technique for non-invasively measuring blood pressure in which pressure is applied to a patient's finger in such a manner that arterial pressure may be balanced by a time varying pressure to maintain a constant arterial volume. In a properly fitted and calibrated system, the applied time varying pressure is equal to the arterial blood pressure in the finger. The applied time varying pressure may be measured to provide a reading of the patient's arterial blood pressure.

This may be accomplished by a finger cuff that is arranged around a finger of a patient. The finger cuff may include an infrared light source, an infrared sensor, and an inflatable bladder. The infrared light may be sent through the finger in which a finger artery is present. The infrared sensor picks up the infrared light and the amount of infrared light registered by the sensor may be inversely proportional to the artery diameter and indicative of the pressure in the artery.

In the finger cuff implementation, by inflating the bladder in the finger cuff, a pressure is exerted on the finger artery. If the pressure is high enough, it will compress the artery and the amount of light registered by the sensor will increase. The amount of pressure necessary in the inflatable bladder to compress the artery is dependent on the blood pressure. By controlling the pressure of the inflatable bladder such that the diameter of the finger artery is kept constant, the blood pressure may be monitored in very precise detail as the pressure in the inflatable bladder is directly linked to the blood pressure.

Even so, however, presently utilized finger cuff implementations may not provide accurate measurement of blood pressure. For example, it is known that perfusion conditions of the patient's finger have an impact on the blood pressure measurement. As such, when the patient has poor perfusion (e.g., cold finger), it may take a prolonged time to obtain a stable blood pressure measurement or the system may completely fail to obtain a blood pressure measurement.

### SUMMARY

Embodiments of the invention may relate to a method to measure a patient's blood pressure by a blood pressure measurement system utilizing a finger cuff, in which the finger cuff includes a light emitting diode (LED)—photodiode (PD) pair and a bladder. The method comprises: placing the finger cuff around the patient's finger such that the bladder and the LED-PD pair aid in measuring the patient's blood pressure by the blood pressure measurement system; and applying heat generated by a heating element to the finger of the patient to increase the temperature of the finger, wherein the heating element generates heat at a predetermined temperature. Also, in some embodiments, the heating element also includes warming the hand and the wrist, as will be described.

### BRIEF DESCRIPTION OF THE DRAWINGS

FIG. 1 is a diagram of an example of a blood pressure measurement system according to one embodiment.

FIG. 2 is a diagram illustrating an example of a finger cuff with a heating element according to one embodiment.

FIG. 3 is a block diagram illustrating a blood pressure measurement system according to one embodiment.

FIG. 4 is a diagram illustrating an example of a finger cuff and a heating device according to one embodiment.

FIG. 5 is a diagram illustrating an example of a finger cuff and a heating fixture according to one embodiment.

FIG. 6 is a diagram illustrating an example of a finger cuff and a heating glove according to one embodiment.

### DETAILED DESCRIPTION

Embodiments of the invention may relate to a method to measure a patient's blood pressure by a blood pressure measurement system utilizing a finger cuff, in which the finger cuff includes a light emitting diode (LED)—photodiode (PD) pair and a bladder. The method may comprise: placing the finger cuff around the patient's finger such that the bladder and the LED-PD pair aid in measuring the patient's blood pressure by the blood pressure measurement system utilizing volume clamping; and applying heat generated by a heating element to the finger of the patient to increase the temperature of the finger, wherein the heating element generates heat at a predetermined temperature. As will be described in more detail hereafter, the heating element may be included in the finger cuff. Also, as will be described in more detail hereafter, a heating device may also be used, alone, or in addition to the heating element of the finger cuff to generate and apply heat to the hand of the patient at a predetermined temperature to increase the temperature of the hand. Additionally, as will be described in more detail hereafter, a heating fixture may also be used, alone, or in addition to the heating element of the finger cuff to generate and apply heat to the hand and the wrist of the patient at a predetermined temperature to increase the temperature of the hand and the wrist.

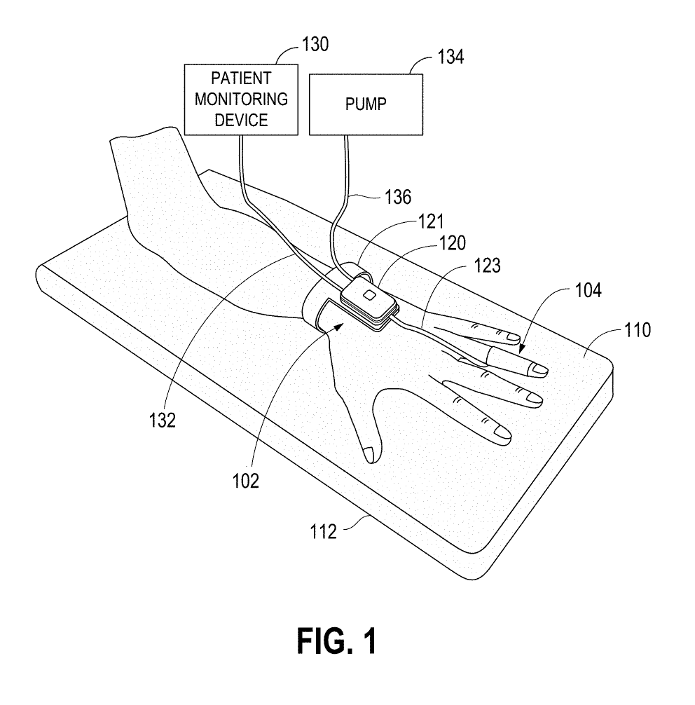

With reference to FIG. 1, which illustrates an example of a blood pressure measurement system according to one embodiment, a blood pressure measurement system 102 that includes a finger cuff 104 that may be attached to a patient's finger and a blood pressure measurement controller 120, which may be attached to the patient's body (e.g., a patient's wrist or hand) is shown.

The blood pressure measurement system 102 may further be connected to a patient monitoring device 130, and, in some embodiments, a pump 134. Further, finger cuff 104 may include a bladder (not shown) and an LED-PD pair (not shown), which are conventional for finger cuffs. In some embodiments, finger cuff 104 may further include a heating element (not shown) integrated with finger cuff 104, as will be discussed in more detail herein below.

In one embodiment, the blood pressure measurement system 102 may include a pressure measurement controller 120 that includes: a small internal pump, a small internal valve, a pressure sensor, and control circuitry. In this embodiment, the control circuitry may be configured to: control the pneumatic pressure applied by the internal pump to the bladder of the finger cuff 104 to replicate the patient's blood pressure based upon measuring the pleth signal received from the LED-PD pair of the finger cuff 104. Further, the control circuitry may be configured to: control the opening of the internal valve to release pneumatic pressure from the bladder; or the internal valve may simply be an orifice that is not controlled. Additionally, the control circuitry may be configured to: measure the patient's blood pressure by monitoring the pressure of the bladder based upon the input from a pressure sensor, which should be the same as patient's blood pressure, and may display the patient's blood pressure on the patient monitoring device 130.

In another embodiment, a conventional pressure generating and regulating system may be utilized, in which, a pump 134 is located remotely from the body of the patient. In this embodiment, the blood pressure measurement controller 120 receives pneumatic pressure from remote pump 134 through tube 136 and passes on the pneumatic pressure through tube 123 to the bladder of finger cuff 104. Blood pressure measurement device controller 120 may also control the pneumatic pressure (e.g., utilizing a controllable valve) applied to the finger cuff 104 as well as other functions. In this example, the pneumatic pressure applied by the pump 134 to the bladder of finger cuff 104 to replicate the patient's blood pressure based upon measuring the pleth signal received from the LED-PD pair of the finger cuff 104 and measuring the patient's blood pressure by monitoring the pressure of the bladder may be controlled by the blood pressure measurement controller 120 and/or a remote computing device and/or the pump 134 and/or the patient monitoring device 130 to implement the volume clamping method. In some embodiments, a blood pressure measurement controller 120 is not used at all and there is simply a connection from tube 136 from a remote pump 134 including a remote pressure regulatory system to finger cuff 104, and all processing for the pressure generating and regulatory system, data processing, and display is performed by a remote computing device.

Continuing with this example, as shown in FIG. 1, a patient's hand may be placed on the face 110 of an arm rest 112 for measuring a patient's blood pressure with the blood pressure measurement system 102. The blood pressure measurement controller 120 of the blood pressure measurement system 102 may be coupled to a bladder of the finger cuff 104 in order to provide pneumatic pressure to the bladder for use in blood pressure measurement. Blood pressure measurement controller 120 may be coupled to the patient monitoring device 130 through a power/data cable 132. Also, in one embodiment, as previously described, in a remote implementation, blood pressure measurement controller 120 may be coupled to a remote pump 134 through tube 136 to receive pneumatic pressure for the bladder of the finger cuff 104. The patient monitoring device 130 may be any type of medical electronic device that may read, collect, process, display, etc., physiological readings/data of a patient including blood pressure, as well as any other suitable physiological patient readings. Accordingly, power/data cable 132 may transmit data to and from patient monitoring device 130 and also may provide power from the patient monitoring device 130 to the blood pressure measurement controller 120 and finger cuff 104.

As can be seen in FIG. 1, in one example, the finger cuff 104 may be attached to a patient's finger and the blood pressure measurement controller 120 may be attached on the patient's hand or wrist with an attachment bracelet 121 that wraps around the patient's wrist or hand. The attachment bracelet 121 may be metal, plastic, Velcro, etc. It should be appreciated that this is just one example of attaching a blood pressure measurement controller 120 and that any suitable way of attaching a blood pressure measurement controller to a patient's body or in close proximity to a patient's body may be utilized and that, in some embodiments, a blood pressure measurement controller 120 may not be used at all. It should further be appreciated that the finger cuff 104 may be connected to a blood pressure measurement controller described herein, or a pressure generating and regulating system of any other kind, such as a conventional pressure generating and regulating system that is located remotely from the body of the patient (e.g., a pump 134 located remotely from a patient). Any kind of pressure generating and regulating system can be used, including but not limited to the blood pressure measurement controller, and may be described simply as a pressure generating and regulating system that may be used with a finger cuff 104 including an LED-PD pair and a bladder to implement the volume clamping method. Further, any sort of suitable finger cuff 104 including an LED-PD pair and a bladder may be utilized to implement the volume clamping method. For example, finger cuff 104 may include a suitable wrapping portion that wraps around the patient's finger (e.g., being of flexible material with a Velcro clamping system) that can be connected around the patient's finger and that includes an LED-PD pair and a bladder to aid in measuring the patient's blood pressure by the blood pressure measurement system utilizing the volume clamping method.

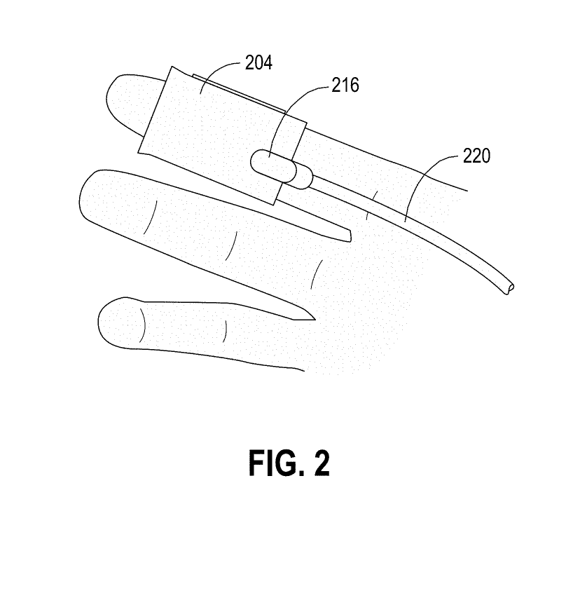

FIG. 2 is a diagram illustrating an example of a finger cuff with a heating element according to one embodiment. As can be seen in FIG. 2, finger cuff 204 having a wrapping portion with a connection portion may be wrapped around and connected to a patient's finger. In one embodiment, finger cuff 204 may include a heating element 216 that generates heat. For example, heating element 216 may receive electrical current from a voltage source (not shown) via cable 220 and convert the electrical current into heat (e.g., through a process of resistive or Joule heating) at a specific temperature (e.g., 35° C.). Heat generated from heating element 216 may be transferred or applied to the patient's finger through the finger cuff 204 itself to the patient's finger, thereby increasing the temperature of the finger (e.g., warming up the finger). In some embodiments, heating element 216 may be attached to an outer surface of finger cuff 204 such that it does not touch the patient's finger but heats the finger cuff 204 and thereby the patient's finger. In other embodiments, heating element 216 may be integrated within finger cuff 204 and may heat the finger cuff 204 and thereby the patient's finger, but does not directly abut against and does not touch the patient's finger. In some embodiments, heating element 216 may be integrated within the finger cuff 204 and may heat the finger cuff 204 and thereby the patient's finger and also may directly touch the patient's finger to heat the patient's finger. In some embodiments, heating element 216 may be covered or wrapped with insulator materials that can withstand heat such as fabric, rubber, heat resistance plastic, etc.

The voltage source, for example, may be an alternating current (AC) voltage source (e.g., wall outlet) or a direct current (DC) voltage source (e.g., a battery). In some embodiments, heating element 216 may be a metal heating element, ceramic heating element, polymer positive temperature coefficient (PTC) heating element, composite heating element, or a combination thereof.

In various embodiments, having a heating element 216 combined with finger cuff 204 enables reliable blood pressure measurement, particularly for people with poor finger perfusion (e.g., due to cold hand). Further, it has been found that the heat factor increases the temperature of the patient's finger to shorten the time (e.g., within 6 minutes) to achieve a reliable blood pressure measurement. Moreover, the heat factor may also improve patient comfortability. In this way, by utilizing a finger cuff 204 having an LED-PD pair and a bladder, that further includes a heating element 216 to heat the patient's finger, the blood pressure measurement system utilizing the volume clamping method can more efficiently and reliably measure the patient's blood pressure.

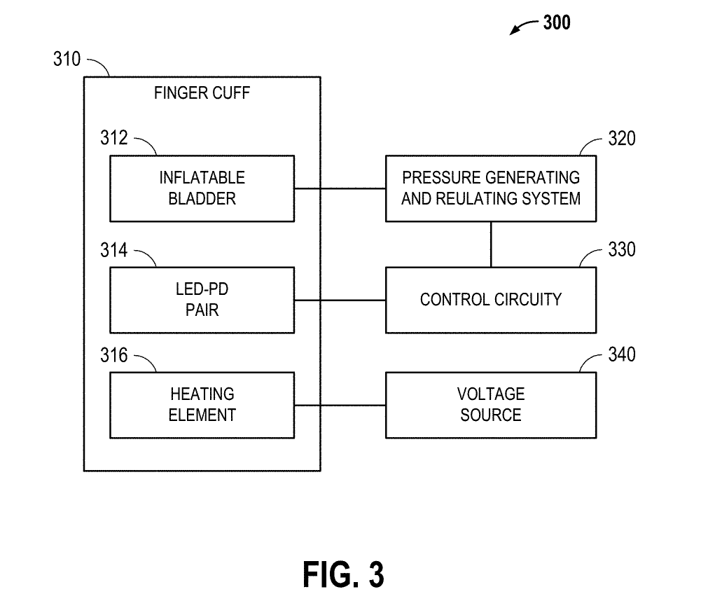

FIG. 3 is a block diagram illustrating a finger cuff and a pressure generating and regulating system. As an example, as shown in FIG. 3, finger cuff 310 may include an inflatable bladder 312, an LED_PD pair 314, and a heating element 316. The inflatable bladder 312 may be pneumatically connected to a pressure generating and regulating system 320. The pressure generating and regulating system 320 and control circuitry 330 may generate, measure, and regulate pneumatic pressure that inflates or deflates the inflatable bladder 312, and may further comprise such elements as a pump, a valve, a pressure sensor, and/or other suitable elements, as previously described. In particular, pressure generating and regulating system 320 in cooperation with control circuitry 330 may be configured to implement a volume clamp method with the finger cuff 310 by: applying pneumatic pressure to the inflatable bladder 312 of the finger cuff 310 to replicate the patient's blood pressure based upon measuring the pleth signal received from the LED-PD pair 314 of the finger cuff 104; and measuring the patient's blood pressure by monitoring the pressure of the inflatable bladder 312 based upon input from a pressure sensor, which should be the same as patient's blood pressure, and may further command the display of the patient's blood pressure on the patient monitoring device. Further, the heating element 316 may generate heat at a specific temperature from electric current received from voltage source 340. For example, by way of resistive heating, heating element 316 may convert the electric current from the voltage source 340 into heat at a specific temperature, with the heat being transferred to the patient's finger through the finger cuff 310, as previously discussed. As has been described, by utilizing a finger cuff 310 that further includes a heating element 316 to heat the patient's finger, the blood pressure measurement system utilizing the volume clamping method can more efficiently and reliably measure the patient's blood pressure. Additionally, although not particularly shown in FIG. 3, in some embodiments, control circuitry 330 may also be connected to an indicator with control circuitry 330 instructing the indicator to alert a healthcare provider (e.g., a nurse), to use a heating device or fixture (as discussed in more detail herein below) to increase the patient's hand temperature for a better signal from the LED-PD pair 314.

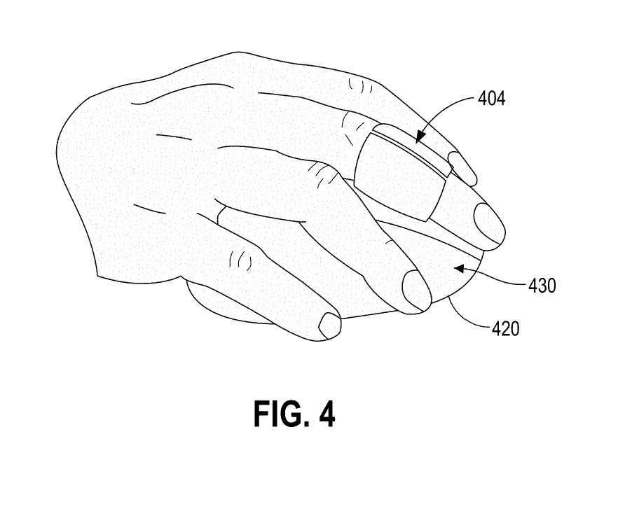

With additional reference to FIG. 4, FIG. 4 is a diagram illustrating an example of a finger cuff and a heating device according to one embodiment. Referring to FIG. 4, a finger cuff 404 is placed around a finger (e.g., a middle finger as shown) of a patient and the patient's hand rests on a heating device 420. Heating device 420 may include a heating element located within the heating device 420 that emits heat through a top surface 430 of heating device 420 and transfers the heat to the patient's hand and finger, thereby increasing the temperature of the patient's hand and finger so that a reliable blood pressure measurement can be obtained using finger cuff 404. Therefore, in this embodiment, the finger cuff 404 is placed around the patient's finger such that the bladder and the LED-PD pair aid in measuring the patient's blood pressure by the blood pressure measurement system using the volume clamp method; and heat generated by the heating device 420 is applied to the hand and finger of the patient at a predetermined temperature to increase the temperature of the hand and finger.

In some embodiments, heating device 420 may be approximately the size of a human's palm and the top surface 430 may be convex (e.g., similar to a computer mouse), such that the palm and fingers of the patient may comfortably rest on heating device 420. Similar to the heating element 216 of FIG. 2, in some embodiments, the heating device 420 may include a heating element that may be a metal heating element, ceramic heating element, polymer positive temperature coefficient (PTC) heating element, composite heating element, or a combination thereof. Further, similar to the heating element 216 of FIG. 2, in some embodiments, the heating element of heating device 420 may receive electrical current from a voltage source and convert the electrical current into heat (e.g., through a process of resistive or Joule heating) at a specific temperature (e.g., 35° C.) and the heat generated from the heating element may be transferred or applied to the patient's hand and finger through the top surface 430 of the heating device 420, thereby increasing the temperature of the finger (e.g., warming up the finger). This heating device 420 implementation may utilized in conjunction with previously described finger cuff 204 having a heating element or a regular finger cuff without its own heating element. By utilizing the heating device 420 implementation to heat the patient's hand and finger, with a finger cuff 404 having an LED-PD pair and a bladder, the blood pressure measurement system utilizing the volume clamping method can more efficiently and reliably measure the patient's blood pressure. In particular, the heating device 420 implementation enables reliable blood pressure measurement, particularly for people with poor finger perfusion (e.g., due to cold hand), which is achievable in a relatively short period of time.

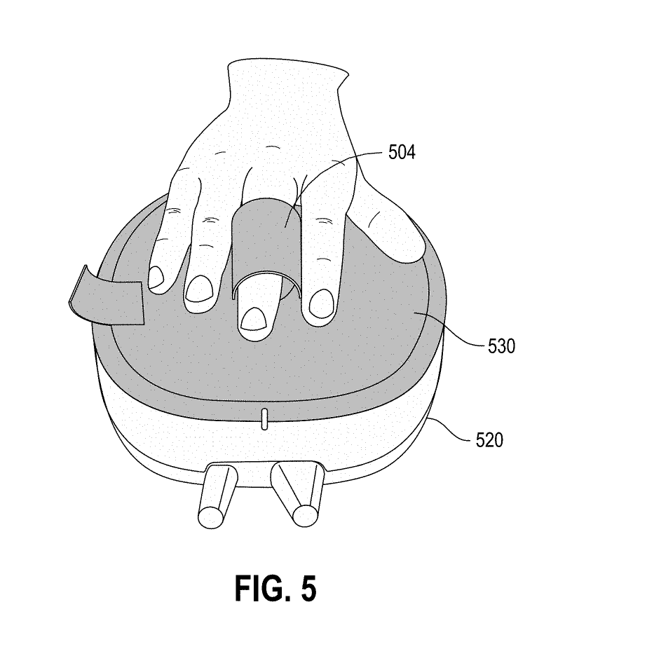

FIG. 5 is a diagram illustrating an example of a finger cuff and a heating fixture according to one embodiment. Referring to FIG. 5, a finger cuff 504 is placed around a finger of a patient. Heating fixture 520 may include a heating element that emits heat at a specific temperature through a pad 530 to the patient's hand, wrist and finger resting on pad 530. The pad 530 may be removably attached onto a top surface of heating fixture 520. The heat is transferred to the patient's hand, wrist, and finger, through the pad 530 thereby increasing the temperature of the hand, wrist, and finger. The increased temperature in the patient's hand, wrist, and finger allows for a reliable blood pressure measurement to be obtained in a relatively short period of time (e.g., within 6 minutes) using finger cuff 504. Therefore, in this embodiment, the finger cuff 504 is placed around the patient's finger such that the bladder and the LED-PD pair aid in measuring the patient's blood pressure by the blood pressure measurement system using the volume clamp method; and heat generated by the heating element of the heating fixture 520 is transferred through the pad 530 to the hand, wrist, and finger of the patient at a predetermined temperature to increase the temperature of the hand, wrist and finger. It should be appreciated that the hand, wrist, and finger are described as resting on the pad, but that the wrist may not necessarily rest on the pad and only the hand and finger may rest on the pad.

Similar to the heating element 216 of FIG. 2, in some embodiments, the heating element of heating fixture 520 may be a metal heating element, ceramic heating element, polymer positive temperature coefficient (PTC) heating element, composite heating element, and combinations thereof. In some embodiments, pad 530 is formed with insulator materials such as fabric, rubber, heat resistance plastic, etc., for example, to cover the heating element. In many embodiments, finger cuff 504 and heating fixture 520 are separate units and operate independently. Alternatively, however, in some embodiments finger cuff 504 may be integrated onto heating fixture 520 such that a patient may simply slide his/her finger through finger cuff 504 and rest his/her hand and/or wrist on heating fixture 520.

Additionally, similar to the heating element 216 of FIG. 2, in some embodiments, the heating element of heating fixture 520 may receive electrical current from a voltage source and convert the electrical current into heat (e.g., through a process of resistive or Joule heating) at a specific temperature (e.g., 35° C.) and the heat generated from heating element may be transferred or applied to the patient's hand, wrist, and finger through the pad 530 of the heating fixture 520, thereby increasing the temperature of the finger (e.g., warming up the finger). This heating fixture 520 implementation may utilized in conjunction with previously described finger cuff 204 having a heating element or a regular finger cuff without its own heating element. By utilizing the heating fixture 520 implementation to heat the patient's hand, wrist, and finger, with a finger cuff 504 having an LED-PD pair and a bladder, the blood pressure measurement system utilizing the volume clamping method can more efficiently and reliably measure the patient's blood pressure. In particular, the heating fixture 520 implementation enables reliable blood pressure measurement, particularly for people with poor finger perfusion (e.g., due to cold hand), which is achievable in a relatively short period of time.

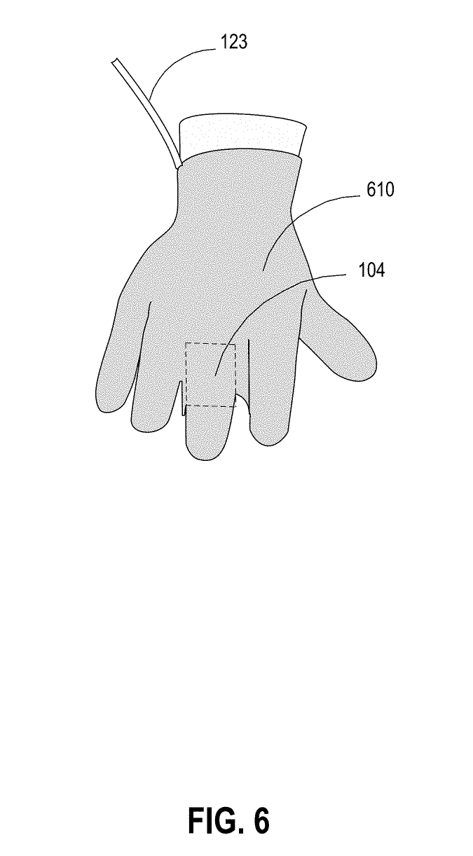

FIG. 6 is a diagram illustrating an example of a finger cuff and a heating glove or mitten according to one embodiment. Referring to FIG. 6, finger cuff 104 is placed around one of the patient's finger (in this example, a middle finger as shown with dashed lines). The patient's hand (with finger cuff 104 on one of the fingers) is then placed inside a heating glove 610 with heating capability. For example, the heating glove 610 may include a heating element that emits heat at a specific temperature. Heat generated from the heating element may be transferred or applied to the hand (or one or more fingers) of the patient through the heating glove 610 itself, thereby increasing the temperature of the hand or finger(s). It should be appreciated that while FIG. 6 illustrates the heating glove 610, in some embodiments, the heating glove 610 may instead be a mitten or any form of a hand wrap.

In some embodiments, the heating element may be attached to an outer surface of the heating glove 610 such that it does not touch the patient's hand or finger(s) but heats the heating glove 610 and thereby the patient's hand or finger(s). In other embodiments, the heating element may be integrated within the heating glove 610 and may heat the heating glove 610, and thereby the patient's hand or finger(s), but does not directly abut against and does not touch the patient's hand or finger(s). In some embodiments, the heating element may be integrated within the heating glove 610 and may heat the heating glove 610, and thereby the patient's hand or finger(s), and also may directly touch the patient's hand or finger(s) to heat the patient's hand or finger(s). In some embodiments, the heating glove 610 may be fabricated using insulator materials that can withstand heat such as fabric, rubber, heat resistance plastic, etc.

As previously described, finger cuff 104 is placed around a finger of a patient (in this example, the middle finger). By placing the patient's hand inside the heating glove 610, the increased temperature in the patient's hand or finger(s) allows for a reliable blood pressure measurement to be obtained in a relatively short period of time (e.g., within 6 minutes) using finger cuff 104. Therefore, in this embodiment, the finger cuff 104 is placed around the patient's finger such that the bladder (which receives pneumatic pressure through tube 123) and the LED-PD pair aid in measuring the patient's blood pressure by the blood pressure measurement system using the volume clamp method; and heat generated by the heating element is transferred through the heating glove 610 to the hand or finger(s) of the patient at a predetermined temperature to increase the temperature of the hand or finger(s). Also, it should appreciated that this is just an example of a glove or mitten with a finger cuff, and any sort of glove or mitten and finger cuff implementation may be utilized. For example, in some embodiments, the glove or mitten 610 may not include the heating element. Instead, the glove or mitten 610 may serve to trap heat from the patient's hand, thereby warming the patient's hand or finger(s).

In other embodiments, heat may be applied to the hand of the patient from a device in a blood pressure measurement system (e.g., blood pressure measurement system 102 of FIG. 1). For example, a pump (e.g., pump 134 of FIG. 1) or control unit (e.g., controller 120 of FIG. 1) may be placed underneath the patient's hand to utilize the generated heat from such device in order to increase the temperature of the patient's hand. In some embodiments, the heating element utilized may be chemical(s) that employ a one-time exothermic chemical reaction, which produces heat for a certain period of time.

It should be appreciated that aspects of the invention previously described may be implemented in conjunction with the execution of instructions by processors, circuitry, controllers, control circuitry, etc. As an example, control circuitry may operate under the control of a program, algorithm, routine, or the execution of instructions to execute methods or processes in accordance with embodiments of the invention previously described. For example, such a program may be implemented in firmware or software (e.g. stored in memory and/or other locations) and may be implemented by processors, control circuitry, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A method to measure a patient's blood pressure by a blood pressure measurement system utilizing a finger cuff, the finger cuff including a light emitting diode (LED)—photodiode (PD) pair and a bladder, the method comprising:
placing the finger cuff around a patient's finger such that the bladder and the LED-PD pair aid in measuring the patient's blood pressure by the blood pressure measurement system; and
applying heat generated by a heating element to the finger of the patient to increase the temperature of the finger, wherein the heating element generates heat at a predetermined temperature.

2. The method of claim 1, wherein applying the heat generated from the heating element to the finger of the patient comprises:
providing, by a voltage source, an electrical current to the heating element;
converting, by the heating element, the electrical current into heat; and
transferring, by the heating element, the converted heat to the finger of the patient to increase the temperature of the finger.

3. The method of claim 1, wherein applying the heat generated from the heating element to the finger of the patient is performed while obtaining the measurement of the patient's blood pressure.

4. The method of claim 1, wherein the heating element is located within the finger cuff or at an outer surface of the finger cuff and does not abut directly against the finger of the patient.

5. The method of claim 1, wherein the predetermined temperature is approximately 35 degrees Celsius.

6. The method of claim 1, wherein the heating element includes one or more of the following: a metal heating element, ceramic heating element, polymer positive temperature coefficient (PTC) heating element, composite heating element, or chemical(s) that employ an exothermic chemical reaction.

7. The method of claim 1, wherein the blood pressure measurement is obtained within a relatively short period of time.

8. The method of claim 1, wherein subsequent to placing the finger cuff around the patient's finger, placing the patient's hand with the finger cuff around the patient's finger inside a glove, wherein the heating element is located within the heating glove or at an outer surface of the heating glove and does not abut directly against the hand and fingers of the patient.

9. A method to measure a patient's blood pressure by a blood pressure measurement system utilizing a finger cuff, the finger cuff including a light emitting diode (LED)—photodiode (PD) pair and a bladder, the method comprising:
placing the finger cuff around the patient's finger such that the bladder and the LED-PD pair aid in measuring the patient's blood pressure by the blood pressure measurement system; and
applying heat generated by a heating device to the hand of the patient at a predetermined temperature to increase the temperature of the hand.

10. The method of claim 9, wherein the hand rests on the heating device, and wherein the heating device includes a heating element that generates heat at the predetermined temperature.

11. The method of claim 10, wherein applying heat generated from the heating device to the hand of the patient at the predetermined temperature comprises:
providing, by a voltage source, an electrical current to the heating element;
converting, by the heating element, the electrical current into heat; and
transferring, by the heating element, the converted heat to the hand of the patient to increase the temperature of the hand.

12. The method of claim 10, wherein applying heat generated from the heating device to the hand of the patient is performed while obtaining the measurement of the patient's blood pressure.

13. The method of claim 10, wherein the heating element includes one or more of the following: a metal heating element, ceramic heating element, polymer positive temperature coefficient (PTC) heating element, composite heating element, or chemical(s) that employ an exothermic chemical reaction.

14. The method of claim 9, wherein the predetermined temperature is approximately 35 degrees Celsius.

15. The method of claim 9, wherein the blood pressure measurement is obtained within a relatively short period of time.

16. A method to measure a patient's blood pressure by a blood pressure measurement system utilizing a finger cuff, the finger cuff including a light emitting diode (LED)—photodiode (PD) pair and a bladder, the method comprising:
placing the finger cuff around the patient's finger such that the bladder and the LED-PD pair aid in measuring the patient's blood pressure by the blood pressure measurement system; and
transferring heat generated from a heating fixture to the hand and wrist of the patient at a predetermined temperature to increase the temperature of the hand and wrist.

17. The method of claim 16, wherein the heating fixture includes a heating element that generates heat at the predetermined temperature and a pad that is removably attached onto a top surface of the heating fixture, wherein the hand and wrist of the patient rest on the pad.

18. The method of claim 17, wherein the heating element includes one or more of the following: a metal heating element, ceramic heating element, polymer positive temperature coefficient (PTC) heating element, composite heating element, and chemical(s) that employ an exothermic chemical reaction.

19. The method of claim 16, wherein the predetermined temperature is approximately 35 degrees Celsius.

20. The method of claim 16, wherein the blood pressure measurement is obtained within a relatively short period of time.
